(19) United States
(12) Patent Application Publication
(10) Pub. No.: US 2026/0193160 A1
(43) Pub. Date: Jul. 9, 2026

(54) NEW METHOD FOR THE OZONOLYTIC SYNTHESIS OF HIGH MELTING DICARBOXYLIC ACIDS AND OXO-ACIDS

(71) Applicant: P2 SCIENCE, INC., Woodbridge, CT (US)

(72) Inventors: Patrick FOLEY, New Haven, CT (US); Yonghua YANG, East Lyme, CT (US)

(21) Appl. No.: 19/132,245

(22) PCT Filed: Nov. 22, 2023
(86) PCT No.: PCT/US2023/080994

§ 371 Date: May 22, 2025

Related U.S. Application Data
(60) Provisional application No. 63/386,640, filed on Dec. 8, 2022.
(60) Provisional application No. 63/483,405, filed on Feb. 6, 2023.

(51) Int. Cl. C07C 51/34; C07C 51/42

(52) U.S. Cl. CPC C07C 51/34; C07C 51/42; C07C 2531/06

(57) ABSTRACT

The present disclosure pertains to improved methods of preparing high melting dicarboxylic acids or oxo-acids (e.g., carboxylic acid/aldehydes) by ozonolysis of their esters. For example, the present disclosure provides methods for preparing brassylic acid by way of the ozonolysis of erucic acid ester, the method comprising the steps of converting the erucic acid to an ester, performing ozonolysis on the ester, and converting the resulting brassylate ester product to brassylic acid.

### CROSS-REFERENCE TO RELATED APPLICATIONS

This is an international application which claims priority to, and the benefit of, U.S. Provisional Application Ser. No. 63/384,640, filed on Nov. 22, 2022, and U.S. Provisional Application Ser. No. 63/483,405, filed on Feb. 6, 2023, the contents of each of which are hereby incorporated by reference in their entireties.

### FIELD OF INVENTION

The present disclosure pertains to improved methods of preparing high melting dicarboxylic acids or oxo-acids (e.g., carboxylic acid/aldehydes) by ozonolysis of their esters. For example, the present disclosure provides methods for preparing brassylic acid by way of the ozonolysis of erucic acid ester, the method comprising the steps of converting the erucic acid to an ester, performing ozonolysis on the ester, and converting the resulting brassylate ester product to brassylic acid.

### BACKGROUND

Ozonolysis is an industrially useful transformation that involves the oxidation of an unsaturated carbon-carbon bond of an alkene using ozone. The reported mechanism (the “Criegee mechanism”) begins with initial formation of a primary ozonide (1,2,3-trioxolane) intermediate which rapidly decomposes into a carbonyl compound and carbonyl oxide compound. This pair of initial intermediates recombine to form a more stable secondary ozonide (1,2,4-trioxolane), a structure featuring a peroxide bridge.

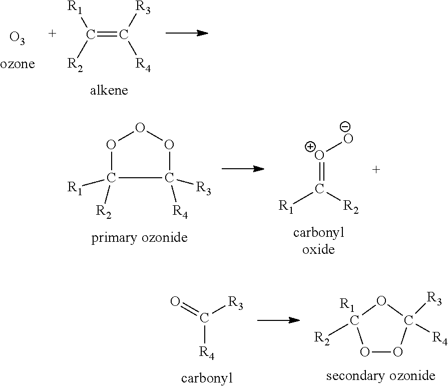

Although more stable than a primary ozonide or a carbonyl oxide, the secondary ozonide is still a high-energy chemical species subject to auto-accelerating thermal decomposition, decomposition to undesirable by-products, and organic peroxide formation (bis-peroxide, poly-peroxide, and hydroperoxide species). Therefore, further reactions must be carefully controlled in order to produce desired carbonyl product in good yield.

Traditionally, the secondary ozonide intermediates are either oxidatively or reductively cleaved to yield carbonyl products. Oxidative cleavage yields carboxylic acid and/or ketone products, while reductive cleavage yields ketone and/or aldehyde products.

The ozonolysis of numerous fatty acids and fatty acid esters have been reported. Under oxidative conditions, the product may be a diacid or a monoacid/monoester, along with a second acid, for example:

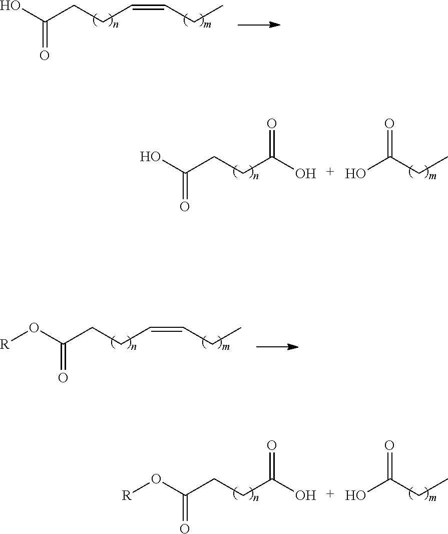

There are numerous monounsaturated fatty acids which could be subjected to such a procedure, including myristolcic acid (n=6, m=3), palmitoleic acid (n=6, m=50), sapicnic acid (n=3, m=8), oleic acid (n=6, m=7), elaidic acid (n=6, m=7), vaccenic acid (n=8, m=5), and erucic acid (n=10, m=7). Simple esters of such fatty acids are also known.

Brassylic acid in particular is a very sought-after fatty acid for its use in flavor and fragrance compositions. It would be very desirable to develop a commercially-feasible, environmentally sustainable method for manufacturing brassylic acid, such as from erucic acid, which is a naturally derivable fatty acid.

The non-ozonolytic oxidative cleavage of erucic acid to form brassylic acid has been reported, e.g., using hydrogen peroxide/sodium tungsten oxide in water; hydrogen peroxide/tungstic acid in tert-butanol; sodium periodate/ruthenium trichloride in acetonitrile/ethyl acetate/water; and hydrogen peroxide in water. See, e.g., Kadyrov et al., Topics in Catalysis, 57 (17-20), 1366-1371 (2014); Kovash et al., ChemSusChem 7 (8): 2289-94 (2014); Zimmerman et al., Tetrahedron Letters 46 (18): 3201-03 (2005); Nieschlag, Henry et al., Industrial & Engineering Chemistry Product Research and Development 6 (2): 120-3 (1967); Kadyrov et al., WO 2015/036342A1; Sato et al., JP2009155302A. Such reagents, especially tungsten, iodine, and ruthenium reagents, are not environmentally friendly, though. Furthermore, hydrogen peroxide is a difficult to handle and potentially quite dangerous to work with.

The non-ozonolytic oxidative cleavage of erucic acid methyl ester to form methyl brassylate has also been reported, using a multi-step process requiring (1) formic acid/hydrogen peroxide in water, (2) sulfuric acid/tert-butanol/water, and (3) oxygen gas with a tungsten and cobalt catalyst in water. See Geng, Wei, et al., Huagong Jinzhan 29 (7): 1320-1323, 1329 (2010).

There has been one report of ozonolytic cleavage of erucic acid methyl ester using an ozone/oxygen gas mixture reacting with methyl erucate in a biphasic tert-amyl alcohol/water mixture. See Matthias et al., EP2573062A1 (2013). However, greater than 95% of the product mixture is nonanal and methyl 13-oxodecanonate, with only miniscule amounts of the more useful oxidative products nonanoic acid and methyl brassylate.

Batch methods for oxidative cleavage of esters are inefficient and potentially dangerous due to the collection of a large amount unstable and potentially explosive reagents and intermediates or by-products in a confined space, usually under high temperature and high-pressure conditions. Ozonolysis, especially under flow conditions (e.g., in a concurrent or countercurrent flow reactor), is a particularly favorable alternative means of carrying out fatty acid or fatty ester oxidative cleavage. Such flow ozonolysis conditions combined with continuous quenching of the ozonide intermediate is particularly safe, economical, and sustainable. See, e.g., U.S. Pat. Nos. 9,035,091; 9,604,898; 10,071,944; 10,428,001; 10,934,239; 10,668,446; 10,696,605. It would be particularly advantageous to perform the continuous ozonolysis and oxidative quenching of erucic acid to form brassylic acid under neat (solvent-free) conditions, as this substantially reduces costs and complexity of the manufacturing operation.

However, the present inventors have unexpectedly discovered that erucic acid is unsuitable to such ozonolytic conditions. First, erucic acid is a high melting solid (molecular weight 338, melting point of about 34° C.) and manufacturing-scale process equipment must be kept at a temperature above 60° C. to reliably maintain the erucic acid in a liquid, flowable state (i.e., not too viscous). Second, when neat erucic acid is subjected to ozonolysis in a flow reactor, the product, brassylic acid (molecular weight 242, melting point about 112° C.) tends to solidify randomly, blocking the flow tubes and potentially causing overpressure and overtemperature conditions which could lead to explosions. This is all the more unexpected because the same problems are not found using the related fatty acid oleic acid (molecular weight 283, melting point 15° C.), to form azelaic acid (molecular weight 188, melting point 111° C.).

There is thus a need for an improved method of preparing brassylic acid from erucic acid, especially on a manufacturing scale. There is also a need for applying the same technology to the preparation of similar high-melting point dicarboxylic acids from their fatty acid ester precursors via ozonolysis. Such other dicarboxylic acids include azelaic acid (melting point 106° C.), undecanedioic acid (melting point 110° C.), pentadecanedioic acid (melting point >110° C.), adipic acid (melting point 152° C.), glutaric acid (melting point 95° C.), and pimelic acid (melting point 105° C.).

### BRIEF SUMMARY OF THE INVENTION

The present disclosure provides a method for preparing a high-melting point dicarboxylic acid (e.g., brassylic acid) from a monounsaturated or polyunsaturated fatty acid (e.g., erucic acid), comprising the steps of converting the fatty acid (e.g., erucic acid) to a C₃₋₁₂ fatty acid ester (e.g., a C₃₋₁₂ erucic acid ester), performing ozonolysis with oxidative work-up on the C₃₋₁₂ fatty acid ester to form an acid/ester product (e.g., brassylic acid ester), and converting the acid ester product (e.g., brassylic acid ester) to the dicarboxylic acid (e.g., brassylic acid). In some embodiments, the ozonolysis is carried out using a flow reactor (e.g., a co-current flow reactor, such as a falling film reactor) with continuous oxidative quenching of the ozonide product stream. In some embodiments, the conversion of the acid ester to the dicarboxylic acid is conducted continuously using acid- or base-catalyzed aqueous hydrolysis.

In another embodiment, the present disclosure provides a method for preparing a high-melting point oxo-carboxylic acid (e.g., brassyl aldehyde) from a monounsaturated or polyunsaturated fatty acid (e.g., erucic acid), comprising the steps of converting the fatty acid (e.g., erucic acid) to a C₃₋₁₂ fatty acid ester (e.g., a C₃₋₁₂ erucic acid ester), performing ozonolysis with reductive work-up on the C₃₋₁₂ fatty acid ester to form an aldehyde/ester product (e.g., brassylic aldehyde ester), and converting the aldehyde ester product (e.g., brassyl aldehyde ester) to the oxo-carboxylic acid (e.g., brassyl aldehyde), wherein the ozonolysis is carried out using a flow reactor (e.g., a co-current flow reactor, such as a falling film reactor) with continuous reductive quenching of the ozonide product stream. In some embodiments, the conversion of the aldehyde ester to the oxo-carboxylic acid is conducted continuously using acid- or base-catalyzed aqueous hydrolysis.

In another embodiment, the present disclosure provides a method for preparing a high melting point dicarboxylic acid (e.g., brassylic acid) from a monounsaturated or polyunsaturated fatty acid (e.g., erucic acid), comprising the steps of converting the fatty acid to a C₃₋₁₂ fatty acid ester (e.g., a C₃₋₁₂ erucic acid ester), performing ozonolysis with reductive work-up on the C₃₋₁₂ fatty acid ester to form the oxo-carboxylic acid ester (e.g., brassylic aldehyde ester), and an aldehyde by-product (e.g., nonanal), removing the aldehyde by-product, oxidizing the oxo-carboxylic acid ester (e.g., brassylic aldehyde ester) to form the dicarboxylic acid ester (e.g., brassylic acid ester), and converting the dicarboxylic acid ester to the dicarboxylic acid (acid (e.g., brassylic acid).). In some embodiments, the ozonolysis is carried out using a flow reactor (e.g., a co-current flow reactor, such as a falling film reactor) with continuous reductive quenching of the ozonide product stream. In some embodiments, the conversion of the acid ester to the dicarboxylic acid is conducted continuously using acid- or base-catalyzed aqueous hydrolysis.

### DETAILED DESCRIPTION OF THE INVENTION

The inventors have unexpectedly discovered that using a C₃₋₁₂ erucic acid ester as the starting material for ozonolysis results in the clean, high-yield production of brassylic acid ester, even using neat ester as the reactant, in a falling film reactor. The same benefit can similarly be obtained using other fatty acid esters to generate corresponding dicarboxylic acid products via ozonolysis, wherein said dicarboxylic acids are high melting point dicarboxylic acids.

All melting points referred to herein are melting points at standard atmospheric pressure (1 atmosphere).

As used herein, in terms such as “C₃₋₁₂ fatty acid ester” and “C₃₋₁₂ erucic acid ester,” the C₃₋₁₂″ designation refers to the alcohol-derived moiety, i.e., a C₃₋₁₂ fatty acid ester is the ester product obtained from a C₃₋₁₂ alcohol condensing with a fatty acid of any chain length. Likewise, the term “C₃₋₁₂ erucic acid ester” refers to the ester product between a C₃₋₁₂ alcohol and erucic acid. Thus, in various embodiments, a C₃₋₁₂ fatty acid ester may include any ester having a linear or branched alcoholic moiety of 3-12 carbon atoms, for example, a propyl, isopropyl, n-butyl, isobutyl, sec-butyl, t-butyl, n-pentyl, isopentyl, sec-pentyl, t-pentyl, n-hexyl, n-nonyl, n-decyl, isodecyl, n-undecyl, or n-dodecyl ester. As used herein, the term “isodecyl” refers to any branched 10-carbon alkyl moiety, such as 4-dimethyloctan-2-yl, 2,6-dimethyl-octan-1-yl, 2,6-dimethyloctan-2-yl, 3,7-dimethyloctan-1-yl, and 3,7-dimethyloctan-3-yl. Isopropyl, isobutyl, and isopentyl, have their standard meanings (i.e., isopropyl is 1-methylethyl, isobutyl is 2-methylpropyl, and isopentyl is 3-methylbutyl).

It is further noted that in describing esters, the term “alcohol-derived” with references to the C₃₋₁₂ alkoxylic component of the ester equally applies where the ester is prepared synthetically using a non-alcohol mediated reaction, such as, nucleophilic condensation of the carboxylic acid with an alkene. Thus, for example, an isodecyl ester could be synthesized by reacting a fatty acid with an isodecanol or with an isodecene.

In a first embodiment of the first aspect, the present disclosure therefore provides, a method (Method 1) for preparing a high melting point dicarboxylic acid (e.g., brassylic acid) from a monounsaturated or polyunsaturated fatty acid (e.g., erucic acid), comprising the steps of:
(a) converting the fatty acid to a C₃₋₁₂ fatty acid ester;
(b) performing ozonolysis with oxidative work-up on the C₃₋₁₂ fatty acid ester to form the dicarboxylic acid ester and carboxylic acid by-product;
   and
(c) converting the dicarboxylic acid ester to the carboxylic acid product (e.g., by aqueous hydrolysis).

In further embodiments of the first aspect, the present disclosure provides:
1.1 Method 1, wherein the fatty acid is a monounsaturated fatty acid, optionally cis or trans in configuration at the double bond.
1.2 Method 1, wherein the fatty acid is a polyunsaturated fatty acid, optionally cis or trans in configuration at the double bonds.
1.3 Method 1, or any of 1.1-1.2, wherein the fatty acid has a melting point above 15° C., e.g., above 20° C., or above 25° C., or above 30° C., e.g., up to 60° C.
1.4 Method 1, or any of 1.1-1.3, wherein the dicarboxylic acid product has a melting point above 60° C., e.g., above 70° C., or above 80° C., or above 90° C., e.g., up to 60° C.
1.5 Method 1, or any of 1.1-1.4, wherein the fatty acid is a compound of Formula Ia or Ib:

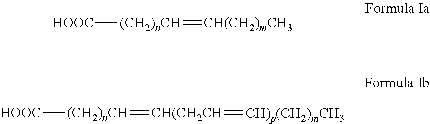

wherein n is an integer from 2 to 13 (e.g., 2, 3, 4, 5, 6, 7, 9, 11, or 13) and m is an integer from 1 to 9 (e.g., 1, 4, 5, or 7), and p is an integer from 1 to 7 (e.g., 1, 2, 3, 4, or 5).
1.6 Method 1, or any of 1.1-1.5, wherein the fatty acid ester is a compound of Formula Ila or IIb:

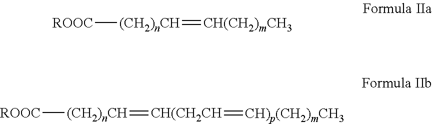

wherein n is an integer from 2 to 13 (e.g., 2, 3, 4, 5, 6, 7, 9, 11, or 13) and m is an integer from 1 to 9 (e.g., 1, 4, 5, or 7), and p is an integer from 1 to 7 (e.g., 1, 2, 3, 4, or 5), and R is a C₃₋₁₂ alkyl (e.g., isopropyl, sec-butyl, 2-ethylhexyl, isodecyl).
1.7 Method 1, or any of 1.1-1.6, wherein the dicarboxylic acid/ester is a compound of Formula III:

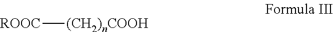

wherein n is an integer from 2 to 13 (e.g., 2, 3, 4, 5, 6, 7, 9, 11, or 13), and R is a C₃₋₁₂ alkyl (e.g., isopropyl, sec-butyl, 2-ethylhexyl, isodecyl).
1.8 Method 1, or any of 1.1-1.7, wherein the dicarboxylic acid is a compound of Formula IV:

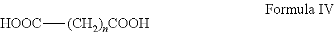

wherein n is an integer from 2 to 13 (e.g., 2, 3, 4, 5, 6, 7, 9, 11, or 13).
1.9 Method 1, or any of 1.1-1.8, wherein the reaction of step (b) further provides a by-product of Formula Va or a mixture of by-products of Formulas Va and Vb:

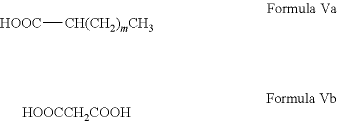

wherein m is an integer from 1 to 9 (e.g., 1, 4, 5, or 7).
1.10 Method 1, or any of 1.1-1.9, wherein R is C₃₋₁₀ alkyl (e.g., isopropyl, sec-butyl, 2-ethylhexyl, isodecyl).
1.11 Method 1, or any of 1.1-1.9, wherein R is C₄₋₈ alkyl (e.g., sec-butyl, 2-ethylhexyl).
1.12 Method 1, or any of 1.1-1.9, wherein R is selected from isopropyl, n-butyl, sec-butyl, isobutyl, tert-butyl, n-pentyl, isopentyl, tert-amyl, n-hexyl, n-heptyl, 2-ethylheptyl, n-octyl, 2-ethylhexyl, n-nonyl, n-decyl, isodecyl (e.g., 2,4-dimethyloctan-2-yl, 2,6-dimethyl-octan-1-yl, 2,6-dimethyloctan-2-yl, 3,7-dimethyloctan-1-yl, and 3,7-dimethyloctan-3-yl).
1.13 Method 1, or any of 1.1-1.12, wherein the fatty acid is selected from erucic acid, palmitoleic acid, oleic acid, elaidic acid, vaccenic acid, gondonic acid, paullinic acid, nervonic acid, stearidonic acid, gamma-linolenic acid, eicosapentaenoic acid, arachidonic acid, docosatetraenonic acid, linolenic acid, linoleic acid, and linoleaidic acid.
1.14 Method 1, or any of 1.1-1.12, wherein the fatty acid is selected from erucic acid and paullinic acid.
1.15 Method 1, or any of 1.1-1.12, wherein the fatty acid is selected from palmitoleic acid, oleic acid, elaidic acid, linolenic acid, and linoleic acid.
1.16 Method 1, or any of 1.1-1.12, wherein the fatty acid is erucic acid.
1.17 Method 1, or any of 1.1-1.16, wherein the product dicarboxylic acid is selected from brassylic acid, azelaic acid, adipic acid, glutaric acid, pimelic acid, undecanedioic acid, and pentaundecanedioic acid.
1.18 Method 1, or any of 1.1-1.16, wherein the product dicarboxylic acid is selected from brassylic acid and azelaic acid.
1.19 Method 1, or any of 1.1-1.16, wherein the product is brassylic acid.
1.20 Method 1, or any of 1.1-1.19, wherein the carboxylic acid by-product(s) are selected from nonanoic acid, heptanoic acid, malonic acid, propionic acid, caproic acid, or mixtures thereof.
1.21 Method 1, or any of 1.1-1.20, wherein the step (b) ozonolysis comprises (1) treating the fatty acid ester (e.g., erucic acid ester) with gaseous ozone in a carrier gas in a flow reactor, followed by (2) continuous quenching of the flow reactor product stream under oxidative conditions.
1.22 Method 1.21, wherein the carrier gas is oxygen, nitrogen, or air.
1.23 Method 1.22, wherein the carrier gas is air (e.g., with 2.5-5% ozone in air).
1.24 Any of Methods 1.21-1.23, wherein the step (b)(1) is carried out in a flow reactor, such as a co-current flow reactor, e.g., a falling film reactor, for example, as described in U.S. Pat. Nos. 10,071,944, 10,428,001, 10,934,239, or U.S. Pat. No. 10,668,446, the contents of each of which are hereby incorporated by reference in their entireties.
1.25 Any of Methods 1.21-1.24, wherein the fatty acid ester (e.g., erucic acid ester) of step (a) is introduced into the reactor of step (b)(1) as an aqueous emulsion consisting of the neat fatty acid ester and aqueous solution (e.g., aqueous buffer or water), for example, without any organic solvents.
1.26 Any of Methods 1.21-1.24, wherein the fatty acid ester (e.g., erucic acid ester) of step (a) is introduced into the reactor of step (b)(1) as the neat fatty acid ester, for example, without any organic solvents or water.
1.27 Method 1.21-1.26, wherein step (b)(2) comprising continuously mixing the output stream from the reactor of step (b)(1) with a quenching solution, e.g., an aqueous solution, organic solution, or aqueous/organic emulsion, comprising an oxidizing agent.
1.28 Method 1.27, wherein the oxidizing agent is air or oxygen over a vanadium or manganese catalyst, such as vanadium oxide or manganese oxide catalyst or a vanadium or manganese complex, optionally in a carboxylic acid solvent (e.g., acetic acid).
1.29 Method 1.27, wherein the oxidizing agent is selected from hydrogen peroxide, organic peroxides (e.g., benzoyl peroxide, methyl ethyl ketone peroxide, tert-butyl hydroperoxide, peracetic acid, trifluoroperacetic acid, peroxymonosulfuric acid, meta-chloroperoxybenzoic acid, potassium monoperoxysulfate).
1.30 Method 1.21-1.29, wherein step (b) comprises water as a co-solvent, optionally, wherein the reaction of step (b) is biphasic (e.g., an emulsion).
1.31 Method 1.21-1.29, wherein step (b) is not biphasic.
1.32 Method 1.21-1.31, wherein step (b) occurs at a temperature from 30 to 100° C.
1.33 Method 1.32, wherein said temperature is between 5° and 90° C. or between 5° and 80° C.
1.34 Method 1, or any of 1.1-1.33, wherein the product of step (b) is purified or partially purified, e.g., by aqueous extractive work-up, by chromatography, by precipitation or crystallization, or by distillation, prior to step (c).
1.35 Method 1.34, wherein the purification provides the intermediate dicarboxylic acid ester (e.g., brassylic acid ester) in a purity of at least 80% for introduction into step (c), e.g., at least 85%, at least 90%, at least 95%, at least 97%, or at least 99% purity, e.g., as measured by HPLC or GC analysis.
1.36 Method 1.34 or 1.35, wherein the purification provides the intermediate dicarboxylic acid ester (e.g., brassylic acid ester) having not more than 20% of fatty acid or fatty acid ester (e.g., erucic acid or erucic acid ester) for introduction into step (c), e.g., not more than 15%, or not more than 10%, or not more than 5%, or not more than 3%, or not more than 2%, or not more than 1%, e.g., as measured by HPLC or GC analysis.
1.37 Method 1.34, 1.35, or 1.36, wherein the purification provides the intermediate dicarboxylic acid ester (e.g., brassylic acid ester) having not more than 20% of the carboxylic acid by-product(s) (e.g., nonanoic acid) for introduction into step (c), e.g., not more than 15%, or not more than 10%, or not more than 5%, or not more than 3%, or not more than 2%, or not more than 1%, e.g., measured by HPLC or GC analysis.
1.38 Method 1, or any of 1.1-1.37, wherein step (a) is carried out by reacting the fatty acid (e.g., erucic acid) with the corresponding C₃₋₁₂ alcohol (R—OH) or C₃₋₁₂ alkene in the presence of an acidic catalyst (e.g., a solid acidic resin).
1.39 Method 1.38, wherein step (a) is carried out by passing a mixture of the erucic acid and the C₃₋₁₂ alcohol (R—OH) or C₃₋₁₂ alkene, either neat or in a solvent, through a flow reactor comprising a solid acidic catalyst.
1.40 Method 1.38 or 1.39, wherein the acidic catalyst is an acidic polymer resin such as Amberlyst or Nafion-H, or a Montmorillonite, or a Zeolite, e.g., Montmorillonite K10, or Amberlyst H-15.
1.41 Method 1, or any of 1.1-1.40, wherein step (c) is carried out by treating the crude or purified or partially purified product from step (b) with water and an acidic or basic catalyst (e.g., aqueous sulfuric acid), optionally with a water-miscible organic solvent.
1.42 Method 1, or any of 1.1-1.40, wherein step (c) is carried out by treating the crude or purified or partially purified product from step (b) with steam under high temperature in presence of a metal or metal oxide catalyst (e.g., 150-500° C., or 150-350° C., or 200-400° C., or 300-400° C.), for example, as described in U.S. Pat. No. 6,646,146, the contents of which are hereby incorporated by reference in its entirety;
1.43 Method 1, or any of 1.1-1.42, wherein the product of step (c) is purified to remove reagents, unreacted starting materials and intermediates, and by-products, e.g., by aqueous extractive work-up, by chromatography, by precipitation, or crystallization, or by distillation, or any combination thereof.
1.44 Method 1.43, wherein the purification provides the dicarboxylic acid product (e.g., brassylic acid product) in a purity of at least 90%, e.g., at least 95%, at least 96%, at least 97%, at least 98%, or at least 99% purity, e.g., as measured by HPLC or GC analysis.
1.45 Method 1, or any of 1.1-1.44, wherein no step of the method comprises the use of any enzyme (e.g., any catalase enzyme).
1.46 Method 1, or any of 1.1-1.45, wherein the product stream from step (b) consists essentially of the dicarboxylic acid ester, the carboxylic acid by-product, and any unreacted fatty acid ester, or carried over fatty acid, for example, brassylic ester, nonanoic acid, and any unreacted erucic acid ester, or carried over unreacted erucic acid.
1.47 Method 1, or any of 1.1-1.46, wherein the product from step (c) consists essentially of the dicarboxylic acid product (e.g., brassylic acid).
1.48 Method 1, or any of 1.1-1.47, wherein the product stream from step (b) and/or step (c) does not contain any reduction products, e.g., the product stream does not contain any aldehydes, alcohols, aldehyde/esters, aldehyde/acids, alcohol/esters, or alcohol acids, such as those derived by reduction of any of the compounds of Formula Ia, Ib, Ila, IIb, III, IV, Va, or Vb.
1.49 Method 1.48, wherein the fatty acid is erucic acid, and the product stream from step (b) and/or step (c) does not contain any reduction products, e.g., the product stream does not contain nonanal, nonanol, 13-oxotridecanoic acid, 13-oxotridecanoate esters, 13-hydroxytridecanoic acid, or 13-hydroxytridecanoate esters.

In some embodiments according to Method 1, or any of 1.1-1.49, the fatty acid is erucic acid and the product dicarboxylic acid is brassylic acid, and the Method 1 is a method of preparing brassylic acid from erucic acid, comprising the steps of:
(a) converting the erucic acid to a C₃₋₁₂ erucic acid ester,

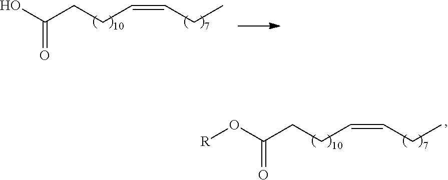

wherein R is a C₃₋₁₂ alkyl (e.g., isopropyl, sec-butyl, 2-ethylhexyl, isodecyl);
(b) performing ozonolysis with oxidative work-up on the C₃₋₁₂ erucic acid ester to form the brassylic acid ester and nonanoic acid by-product,

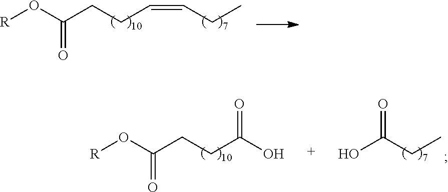

and
(c) converting the brassylic acid ester to brassylic acid (e.g., by aqueous hydrolysis)

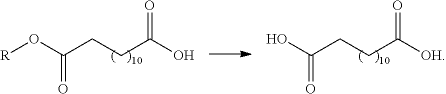

In other embodiments of Method 1, or any of 1.1-1.49, wherein the fatty acid is a different monounsaturated fatty acid, the steps of Method 1 can be drawn as follows:
(a) converting the monounsaturated fatty acid to a C₃₋₁₂ fatty acid ester,

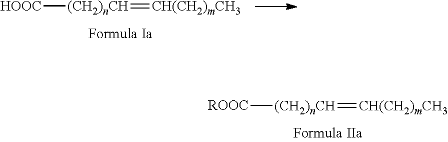

wherein n is an integer from 7 to 13 (e.g., 7, 9, 11, or 13) and m is an integer from 3 to 9 (e.g., 5 or 7), R is a C₃₋₁₂ alkyl (e.g., isopropyl, sec-butyl, 2-ethylhexyl, isodecyl);
(b) performing ozonolysis with oxidative work-up on the C₃₋₁₂ fatty acid ester to form the dicarboxylic acid ester and carboxylic acid by-product,

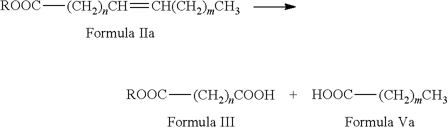

wherein n is an integer from 7 to 13 (e.g., 7, 9, 11, or 13) and m is an integer from 3 to 9 (e.g., 5 or 7), and R is a C₃₋₁₂ alkyl (e.g., isopropyl, sec-butyl, 2-ethylhexyl, isodecyl);
   and
(c) converting the dicarboxylic acid ester to the dicarboxylic acid product (e.g., by aqueous hydrolysis)

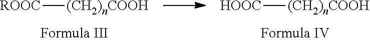

wherein n is an integer from 7 to 13 (e.g., 7, 9, 11, or 13) and m is an integer from 3 to 9 (e.g., 5 or 7), and R is a C₃₋₁₂ alkyl (e.g., isopropyl, sec-butyl, 2-ethylhexyl, isodecyl).

In other embodiments of Method 1, or any of 1.1-1.49, wherein the fatty acid is a polyunsaturated fatty acid, the steps of Method 1 can be drawn as follows:
(a) converting the polyunsaturated fatty acid to a C₃₋₁₂ fatty acid ester,

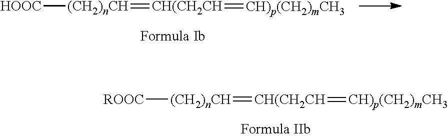

wherein n is an integer from 2 to 9 (e.g., 2, 3, 4, 5, 6, or 7) and m is an integer from 1 to 5 (e.g., 1 or 4), p is an integer from 1 to 7 (e.g., 1, 2, 3, 4, or 5), and R is a C₃₋₁₂ alkyl (e.g., isopropyl, sec-butyl, 2-ethylhexyl, isodecyl);
(b) performing ozonolysis with oxidative work-up on the C₃₋₁₂ fatty acid ester to form the dicarboxylic acid ester and carboxylic acid by-products,

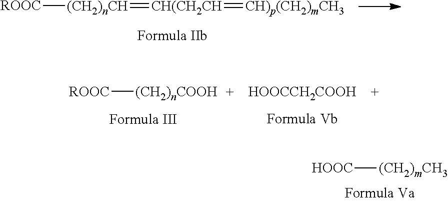

wherein n is an integer from 2 to 9 (e.g., 2, 3, 4, 5, 6, or 7) and m is an integer from 1 to 5 (e.g., 1 or 4), and R is a C₃₋₁₂ alkyl (e.g., isopropyl, sec-butyl, 2-ethylhexyl, isodecyl);
   and
(c) converting the dicarboxylic acid ester to the dicarboxylic acid product (e.g., by aqueous hydrolysis)

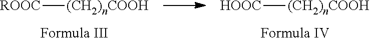

wherein n is an integer from 7 to 13 (e.g., 7, 9, 11, or 13) and m is an integer from 3 to 9 (e.g., 5 or 7), and R is a C₃₋₁₂ alkyl (e.g., isopropyl, sec-butyl, 2-ethylhexyl, isodecyl).

In a second embodiment of the first aspect, the present disclosure therefore provides, a method (Method 2) for preparing a high melting point oxo-carboxylic acid (e.g., brassyl aldehyde) from a monounsaturated or polyunsaturated fatty acid (e.g., erucic acid), comprising the steps of:
(a) converting the fatty acid to a C₃₋₁₂ fatty acid ester;
(b) performing ozonolysis with reductive work-up on the C₃₋₁₂ fatty acid ester to form the oxo-carboxylic acid ester and aldehyde by-product;
   and
(c) converting the oxo-carboxylic acid ester to the oxo-carboxylic acid product (e.g., by aqueous hydrolysis);
   wherein the formation of ozonide intermediate of the ozonolysis step (b) is conducted in a flow reactor.

In further embodiments of the first aspect, the present disclosure provides:
2.1. Method 2, wherein the fatty acid is a monounsaturated fatty acid, optionally cis or trans in configuration at the double bond.
2.2. Method 2, wherein the fatty acid is a polyunsaturated fatty acid, optionally cis or trans in configuration at the double bonds.
2.3. Method 2, or any of 2.1-2.2, wherein the fatty acid has a melting point above 15° C., e.g., above 20° C., or above 25° C., or above 30° C., e.g., up to 60° C.
2.4. Method 2, or any of 2.1-2.3, wherein the dicarboxylic acid product has a melting point above 60° C., e.g., above 70° C., or above 80° C., or above 90° C., e.g., up to 60° C.
2.5. Method 2, or any of 2.1-2.4, wherein the fatty acid is a compound of Formula VIa or VIb:

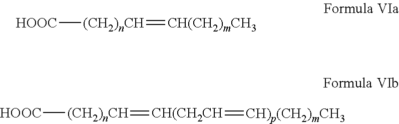

wherein n is an integer from 2 to 13 (e.g., 2, 3, 4, 5, 6, 7, 9, 11, or 13) and m is an integer from 1 to 9 (e.g., 1, 4, 5, or 7), and p is an integer from 1 to 7 (e.g., 1, 2, 3, 4, or 5).
2.6. Method 2, or any of 2.1-2.5, wherein the fatty acid ester is a compound of Formula VIIa or VIIb:

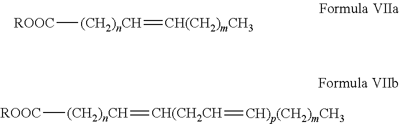

wherein n is an integer from 2 to 13 (e.g., 2, 3, 4, 5, 6, 7, 9, 11, or 13) and m is an integer from 1 to 9 (e.g., 1, 4, 5, or 7), and p is an integer from 1 to 7 (e.g., 1, 2, 3, 4, or 5), and R is a C₃₋₁₂ alkyl (e.g., isopropyl, sec-butyl, 2-ethylhexyl, isodecyl).
2.7. Method 2, or any of 2.1-2.6, wherein the oxo-carboxylic acid/ester is a compound of Formula VIII:

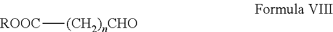

wherein n is an integer from 2 to 13 (e.g., 2, 3, 4, 5, 6, 7, 9, 11, or 13), and R is a C₃₋₁₂ alkyl (e.g., isopropyl, sec-butyl, 2-ethylhexyl, isodecyl).
2.8. Method 2, or any of 2.1-2.7, wherein the oxo-carboxylic acid is a compound of Formula IX:

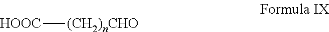

wherein n is an integer from 2 to 13 (e.g., 2, 3, 4, 5, 6, 7, 9, 11, or 13),
2.9. Method 2, or any of 2.1-2.8, wherein the reaction of step (b) further provides a by-product of Formula Xa or a mixture of by-products of Formulas Xa and Xb:

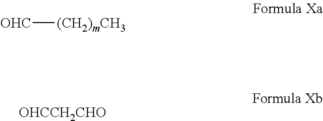

wherein m is an integer from 1 to 9 (e.g., 1, 4, 5, or 7).
2.10. Method 2, or any of 2.1-2.9, wherein R is C₃₋₁₀ alkyl (e.g., isopropyl, sec-butyl, 2-ethylhexyl, isodecyl).
2.11. Method 2, or any of 2.1-2.9, wherein R is C₄₋₈ alkyl (e.g., sec-butyl, 2-ethylhexyl).
2.12. Method 2, or any of 2.1-2.9, wherein R is selected from isopropyl, n-butyl, sec-butyl, isobutyl, tert-butyl, n-pentyl, isopentyl, tert-amyl, n-hexyl, n-heptyl, 2-ethylheptyl, n-octyl, 2-ethylhexyl, n-nonyl, n-decyl, isodecyl (e.g., 2,4-dimethyloctan-2-yl, 2,6-dimethyl-octan-1-yl, 2,6-dimethyloctan-2-yl, 3,7-dimethyloctan-1-yl, and 3,7-dimethyloctan-3-yl).
2.13. Method 2, or any of 2.1-2.12, wherein the fatty acid is selected from erucic acid, palmitoleic acid, oleic acid, elaidic acid, vaccenic acid, gondonic acid, paullinic acid, nervonic acid, stearidonic acid, gamma-linolenic acid, eicosapentaenoic acid, arachidonic acid, docosatetraenonic acid, linolenic acid, linoleic acid, and linoleaidic acid.
2.14. Method 2, or any of 2.1-2.12, wherein the fatty acid is selected from erucic acid and paullinic acid.
2.15. Method 2, or any of 2.1-2.12, wherein the fatty acid is selected from palmitoleic acid, oleic acid, elaidic acid, linolenic acid, and linoleic acid.
2.16. Method 2, or any of 2.1-2.12, wherein the fatty acid is erucic acid.
2.17. Method 2, or any of 2.1-2.16, wherein the product oxo-carboxylic acid is selected from brassyl aldehyde (13-oxotridecanoic acid), 9-oxononanoic acid, 6-oxohexanoic acid, 5-oxopentanoic acid, 7-oxoheptanoic acid, 11-oxoundecanoic acid, and 15-oxopentadecanoic acid.
2.18. Method 2, or any of 2.1-2.16, wherein the product oxo-carboxylic acid is selected from brassyl aldehyde and 9-oxononanoic acid.
2.19. Method 2, or any of 2.1-2.16, wherein the product is brassyl aldehyde.
2.20. Method 2, or any of 2.1-2.19, wherein the aldehyde by-product(s) are selected from nonanal, heptanal, malonaldehyde, propanal, hexanal, or mixtures thereof. 2.21. Method 2, or any of 2.1-2.20, wherein the step (b) ozonolysis comprises (1) treating the fatty acid ester (e.g., erucic acid ester) with gaseous ozone in a carrier gas in a flow reactor, followed by (2) continuous quenching of the flow reactor product stream under reductive conditions.
2.22. Method 2.21, wherein the carrier gas is oxygen, nitrogen, or air.
2.23. Method 2.22, wherein the carrier gas is air (e.g., with 2.5-5% ozone in air).
2.24. Any of Methods 2.21-2.23, wherein the step (b)(1) is carried out in a co-current flow reactor, e.g., a falling film reactor, for example, as described in U.S. Pat. Nos. 10,071,944, 10,428,001, 10,934,239, or U.S. Pat. No. 10,668,446, the contents of each of which are hereby incorporated by reference in their entireties.
2.25. Any of Methods 2.21-2.24, wherein the fatty acid ester (e.g., erucic acid ester) of step (a) is introduced into the reactor of step (b)(1) as an aqueous emulsion consisting of the neat fatty acid ester and aqueous solution (e.g., aqueous buffer or water), for example, without any organic solvents.
2.26. Any of Methods 2.21-2.24, wherein the fatty acid ester (e.g., erucic acid ester) of step (a) is introduced into the reactor of step (b)(1) as the neat fatty acid ester, for example, without any organic solvents or water.
2.27. Method 2.21-2.26, wherein step (b)(2) comprising continuously mixing the output stream from the reactor of step (b)(1) with a quenching solution, e.g., an aqueous solution, organic solution, or aqueous/organic emulsion, comprising a reducing agent.
2.28. Method 2.27, wherein the reducing agent is sodium hydroxymethanesulfinate, hydroxymethanesulfinic acid, a sulfite salt (e.g., sodium sulfite, sodium bisulfite, sodium metabisulfite, potassium sulfite, potassium bisulfite, potassium metabisulfite), sulfur dioxide, sodium dithionite, and dimethyl sulfide.
2.29. Method 2.27, wherein the reducing agent is selected from triphenylphosphine, thiourea, zinc metal, hydrogen gas (e.g., over a palladium, rhodium, or platinum catalyst), or formic acid.
2.30. Method 2.27, wherein the reducing agent is glyoxal (e.g., an aqueous glyoxal solution, such as 40 wt. % aqueous glyoxal).
2.31. Method 2.21-2.30, wherein step (b) comprises water as a co-solvent, optionally, wherein the reaction of step (b) is biphasic (e.g., an emulsion).
2.32. Method 2.21-2.30, wherein step (b) is not biphasic.
2.33. Method 2.21-2.32, wherein step (b) occurs at a temperature from 30 to 100° C.
2.34. Method 2.33, wherein said temperature is between 5° and 90° C. or between 5° and 80° C.
2.35. Method 2, or any of 2.1-2.34, wherein the product of step (b) is purified or partially purified, e.g., by aqueous extractive work-up, by chromatography, by precipitation or crystallization, or by distillation, prior to step (c).
2.36. Method 2.35, wherein the purification provides the intermediate oxo-carboxylic acid ester (e.g., brassyl aldehyde ester) in a purity of at least 80% for introduction into step (c), e.g., at least 85%, at least 90%, at least 95%, at least 97%, or at least 99% purity, e.g., as measured by HPLC or GC analysis.
2.37. Method 2.35 or 2.36, wherein the purification provides the intermediate oxo-carboxylic acid ester (e.g., brassyl aldehyde ester) having not more than 20% of fatty acid or fatty acid ester (e.g., erucic acid or erucic acid ester) for introduction into step (c), e.g., not more than 15%, or not more than 10%, or not more than 5%, or not more than 3%, or not more than 2%, or not more than 1%, e.g., as measured by HPLC or GC analysis.
2.38. Method 2.35, 2.36, or 2.37, wherein the purification provides the intermediate oxo-carboxylic acid ester (e.g., brassyl aldehyde ester) having not more than 20% of the aldehyde by-product(s) (e.g., nonanal) for introduction into step (c), e.g., not more than 15%, or not more than 10%, or not more than 5%, or not more than 3%, or not more than 2%, or not more than 1%, e.g., as measured by HPLC or GC analysis.
2.39. Method 2, or any of 2.1-2.38, wherein step (a) is carried out by reacting the fatty acid (e.g., erucic acid) with the corresponding C₃₋₁₂ alcohol (R—OH) or C₃₋₁₂ alkene in the presence of an acidic catalyst (e.g., a solid acidic resin).
2.40. Method 2.39, wherein step (a) is carried out by passing a mixture of the erucic acid and the C₃₋₁₂ alcohol (R—OH) or C₃₋₁₂ alkene, either neat or in a solvent, through a flow reactor comprising a solid acidic catalyst.
2.41. Method 2.39 or 2.40, wherein the acidic catalyst is an acidic polymer resin such as Amberlyst or Nafion-H, or a Montmorillonite, or a Zeolite, e.g., Montmorillonite K10, or Amberlyst H-15.
2.42. Method 2, or any of 2.1-2.41, wherein step (c) is carried out by treating the crude or purified or partially purified product from step (b) with water and an acidic or basic catalyst (e.g., aqueous sulfuric acid), optionally with a water-miscible organic solvent.
2.43. Method 2, or any of 2.1-2.41, wherein step (c) is carried out by treating the crude or purified or partially purified product from step (b) with steam under high temperature in presence of a metal or metal oxide catalyst (e.g., 150-500° C., or 150-350° C., or 200-400° C., or 300-400° C.), for example, as described in U.S. Pat. No. 6,646,146, the contents of which are hereby incorporated by reference in its entirety;
2.44. Method 2, or any of 2.1-2.43, wherein the product of step (c) is purified to remove reagents, unreacted starting materials and intermediates, and by-products, e.g., by aqueous extractive work-up, by chromatography, by precipitation, or crystallization, or by distillation, or any combination thereof.
2.45. Method 2.44, wherein the purification provides the oxo-carboxylic acid product (e.g., brassyl aldehyde product) in a purity of at least 90%, e.g., at least 95%, at least 96%, at least 97%, at least 98%, or at least 99% purity, e.g., as measured by HPLC or GC analysis.
2.46. Method 2, or any of 2.1-2.45, wherein no step of the method comprises the use of any enzyme (e.g., any catalase enzyme).
2.47. Method 2, or any of 2.1-2.46, wherein the product stream from step (b) consists essentially of the oxo-carboxylic acid ester, the aldehyde by-product, and any unreacted fatty acid ester, or carried over fatty acid, for example, brassyl aldehyde ester, nonanal, and any unreacted erucic acid ester, or carried over unreacted erucic acid.
2.48. Method 2, or any of 2.1-2.47, wherein the product from step (c) consists essentially of the oxo-carboxylic acid product (e.g., brassyl aldehyde).
2.49. Method 2, or any of 2.1-2.48, wherein the product stream from step (b) and/or step (c) does not contain any oxidation products, e.g., the product stream does not contain any carboxylic acids, carboxylic esters, or carboxylic acid/esters, such as those derived by oxidation of any of the compounds of Formula VIa, VIb, VIIa, VIIb, VIII, IX, Xa, or Xb.
2.50. Method 2.49, wherein the fatty acid is erucic acid, and the product stream from step (b) and/or step (c) does not contain any oxidation products, e.g., the product stream does not contain nonanoic acid, brassylic acid, nonanoic acid esters or brassylic acid esters.
2.51. Method 2, or any of 2.1-2.50, wherein the method further comprises the step of isolating the aldehyde by-product (e.g., nonanal) from the crude product, or partially purified product, of step (b) or of step (c).
2.52. Method 2, or any of 2.1-2.50, wherein the step of purifying or partially purifying the crude product of step (b) or step (c) comprises the isolation of the aldehyde by-product (e.g., nonanal).
2.53. Method 2.51 or 2.52, where the aldehyde by-product (e.g., nonanal) is obtained by distillation.

In some embodiments according to Method 2, or any of 2.1-2.53, the fatty acid is erucic acid and the product oxo-carboxylic acid is brassyl aldehyde, and the Method 2 is a method of preparing brassyl aldehyde from erucic acid, comprising the steps of:
(a) converting the erucic acid to a C₃₋₁₂ erucic acid ester,

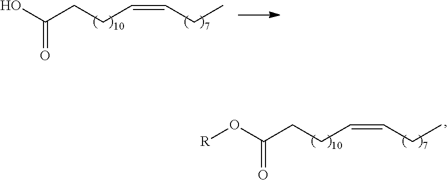

wherein R is a C₃₋₁₂ alkyl (e.g., isopropyl, sec-butyl, 2-ethylhexyl, isodecyl);
(b) performing ozonolysis with reductive work-up on the C₃₋₁₂ erucic acid ester to form the brassyl aldehyde ester and nonanal by-product,

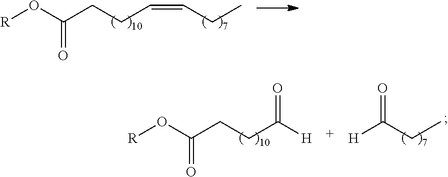

and
(c) converting the brassyl aldehyde ester to brassyl aldehyde (e.g., by aqueous hydrolysis)

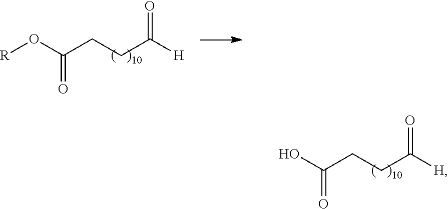

optionally further comprising the isolation and purification of the nonanal by-product.

In other embodiments of Method 2, or any of 2.1-2.53, wherein the fatty acid is a different monounsaturated fatty acid, the steps of Method 2 can be drawn as follows:
(a) converting the monounsaturated fatty acid to a C₃₋₁₂ fatty acid ester,

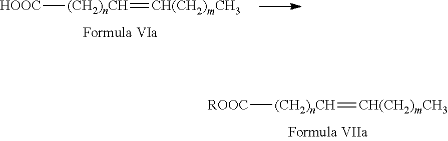

wherein n is an integer from 7 to 13 (e.g., 7, 9, 11, or 13) and m is an integer from 3 to 9 (e.g., 5 or 7), R is a C₃₋₁₂ alkyl (e.g., isopropyl, sec-butyl, 2-ethylhexyl, isodecyl);
(b) performing ozonolysis with reductive work-up on the C₃₋₁₂ fatty acid ester to form the oxo-carboxylic acid ester and aldehyde by-product,

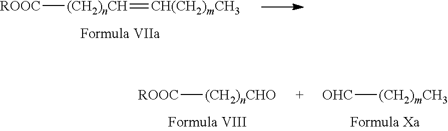

wherein n is an integer from 7 to 13 (e.g., 7, 9, 11, or 13) and m is an integer from 3 to 9 (e.g., 5 or 7), and R is a C₃₋₁₂ alkyl (e.g., isopropyl, sec-butyl, 2-ethylhexyl, isodecyl);
   and
(c) converting the oxo-carboxylic acid ester to the oxo-carboxylic acid product (e.g., by aqueous hydrolysis)

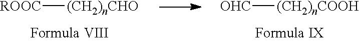

wherein n is an integer from 7 to 13 (e.g., 7, 9, 11, or 13) and m is an integer from 3 to 9 (e.g., 5 or 7), and R is a C₃₋₁₂ alkyl (e.g., isopropyl, sec-butyl, 2-ethylhexyl, isodecyl).

In other embodiments of Method 2, or any of 2.1-2.53, wherein the fatty acid is a polyunsaturated fatty acid, the steps of Method 2 can be drawn as follows:
(a) converting the polyunsaturated fatty acid to a C₃₋₁₂ fatty acid ester,

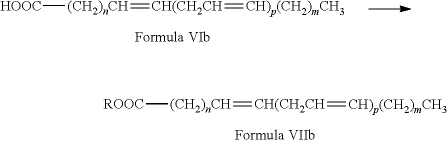

wherein n is an integer from 2 to 9 (e.g., 2, 3, 4, 5, 6, or 7) and m is an integer from 1 to 5 (e.g., 1 or 4), p is an integer from 1 to 7 (e.g., 1, 2, 3, 4, or 5), and R is a C₃₋₁₂ alkyl (e.g., isopropyl, sec-butyl, 2-ethylhexyl, isodecyl);
(b) performing ozonolysis with reductive work-up on the C₃₋₁₂ fatty acid ester to form the oxo-carboxylic acid ester and aldehyde by-products,

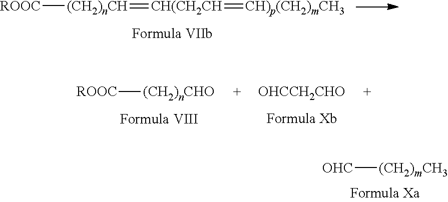

wherein n is an integer from 2 to 9 (e.g., 2, 3, 4, 5, 6, or 7) and m is an integer from 1 to 5 (e.g., 1 or 4), and R is a C₃₋₁₂ alkyl (e.g., isopropyl, sec-butyl, 2-ethylhexyl, isodecyl);
   and
(c) converting the oxo-carboxylic acid ester to the oxo-carboxylic acid product (e.g., by aqueous hydrolysis)

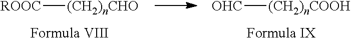

wherein n is an integer from 7 to 13 (e.g., 7, 9, 11, or 13) and m is an integer from 3 to 9 (e.g., 5 or 7), and R is a C₃₋₁₂ alkyl (e.g., isopropyl, sec-butyl, 2-ethylhexyl, isodecyl).

It is understood that the present methods embrace both cis and trans fatty acids as starting materials, although cis fatty acids are preferred because they are more widely dispersed in nature and available from common nuts, seeds, and vegetable oil precursors (e.g., macadamia nuts, guarana, olive oil, pecan oil, canola oil, jojoba oil, wallflower seed, mustard oil, flaxseed, sesame seed, chia seeds, walnuts, hemp seed oil, corn oil, peanut oil, borage oil, black currant oil, and safflower oil. Some of these cis-unsaturated fatty acids are also available from animal products, such as fish oils.

In some embodiments of Method 1, or any of 1.1-1.49, or Method 2, or any of 2.1-2.53, the present disclosure further provides a variation on said methods wherein the initial C₃₋₁₂ fatty acid ester is subjected to ozonolysis with reductive quenching to afford the oxo-carboxylic acid ester and aldehyde products, and after removal of the aldehyde, the oxo-carboxylic acid ester is then oxidized to the dicarboxylic acid ester, optionally followed by hydrolysis to the carboxylic acid product. For example, the present disclosure provides a method (Method 3) for preparing a high melting point dicarboxylic acid (e.g., brassylic acid) from a monounsaturated or polyunsaturated fatty acid (e.g., erucic acid), comprising the steps of:
(a) converting the fatty acid to a C₃₋₁₂ fatty acid ester;
(b) performing ozonolysis with reductive work-up on the C₃₋₁₂ fatty acid ester to form the oxo-carboxylic acid ester and aldehyde by-product;
(c) removing the aldehyde by-product;
(d) oxidizing the oxo-carboxylic acid ester to form the dicarboxylic acid ester; and
(e) converting the dicarboxylic acid ester to the dicarboxylic acid product (e.g., by aqueous hydrolysis);
   optionally, wherein the formation of ozonide intermediate of the ozonolysis step (b) is conducted in a flow reactor.

Thus, in particular embodiments of Method 3, the present disclosure provides a method for preparing a high melting point dicarboxylic acid (e.g., brassylic acid) from a monounsaturated or polyunsaturated fatty acid (e.g., erucic acid), comprising the steps of:
(a) converting the monounsaturated fatty acid to a C₃₋₁₂ fatty acid ester,

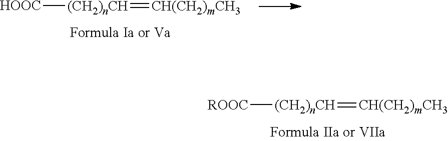

wherein n is an integer from 7 to 13 (e.g., 7, 9, 11, or 13) and m is an integer from 3 to 9 (e.g., 5 or 7), R is a C₃₋₁₂ alkyl (e.g., isopropyl, sec-butyl, 2-ethylhexyl, isodecyl);
(b) performing ozonolysis with reductive work-up on the C₃₋₁₂ fatty acid ester to form the oxo-carboxylic acid ester and aldehyde by-product,

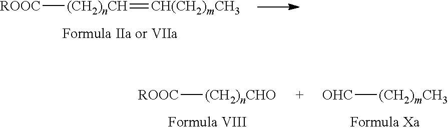

wherein n is an integer from 7 to 13 (e.g., 7, 9, 11, or 13) and m is an integer from 3 to 9 (e.g., 5 or 7), and R is a C₃₋₁₂ alkyl (e.g., isopropyl, sec-butyl, 2-ethylhexyl, isodecyl);
(c) removing the aldehyde product of Formula Xa;
(d) oxidizing the oxo-carboxylic acid ester to form the dicarboxylic acid ester,

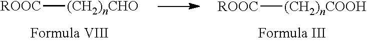

wherein n is an integer from 7 to 13 (e.g., 7, 9, 11, or 13), and R is a C₃₋₁₂ alkyl (e.g., isopropyl, sec-butyl, 2-ethylhexyl, isodecyl); and
(e) converting the dicarboxylic acid ester to the dicarboxylic acid product (e.g., by aqueous hydrolysis)

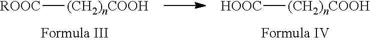

wherein n is an integer from 7 to 13 (e.g., 7, 9, 11, or 13) and m is an integer from 3 to 9 (e.g., 5 or 7), and R is a C₃₋₁₂ alkyl (e.g., isopropyl, sec-butyl, 2-ethylhexyl, isodecyl).

In all respects, the Method 3 may be carried as specifically described in any of the embodiments set forth hereinabove for Methods 1.1-1.49, or Methods 2.1-2.53, as appropriate.

Thus, in particular embodiments of Method 1, or any of 1.1-1.49, or Method 2, or any of 2.1-2.53, as modified above, or Method 3, wherein the fatty acid is erucic acid, the present disclosure provides a method of preparing brassylic acid from erucic acid, comprising the steps of:
(a) converting the erucic acid to a C₃₋₁₂ erucic acid ester,

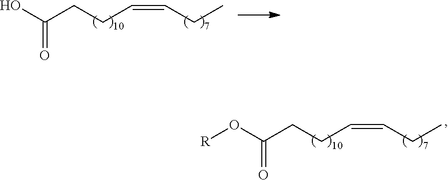

wherein R is a C₃₋₁₂ alkyl (e.g., isopropyl, sec-butyl, 2-ethylhexyl, isodecyl);
(b) performing ozonolysis with reductive work-up on the C₃₋₁₂ erucic acid ester to form the brassyl aldehyde ester and nonanal by-product,

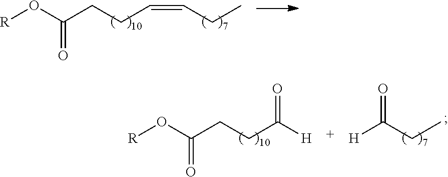

(c) removing the nonanal by-product (e.g., by distillation);
(d) oxidizing the brassyl aldehyde ester to brassylic acid ester (e.g., using vanadium pentoxidc):

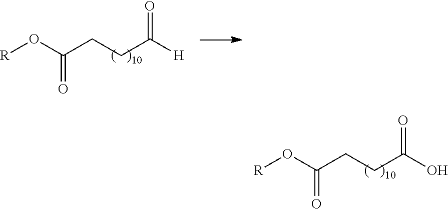

(e) converting the brassylic acid ester to brassylic acid (e.g., by aqueous hydrolysis)

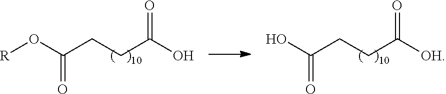

In addition, in any of the preceding embodiments, after the final hydrolysis step, the released C₃₋₁₂ alcohol (R—OH, wherein R is a C₃₋₁₂ alkyl (e.g., isopropyl, sec-butyl, 2-ethylhexyl, isodecyl)), may be recycled such that it is isolated from the hydrolysis step and used as a starting material for the initial ester formation step.

Thus, in one embodiment of the present disclosure, the following process is provided:

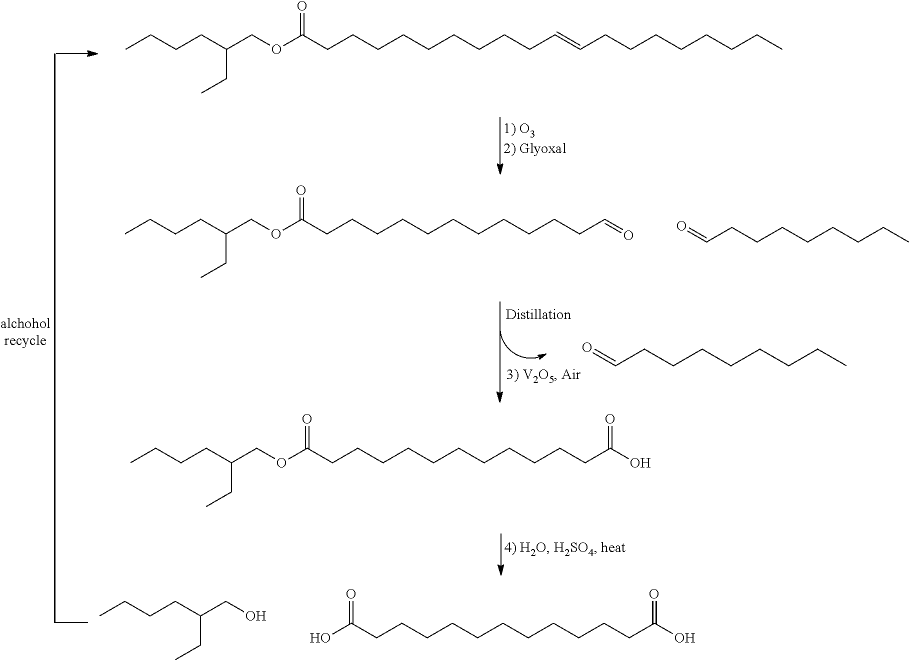

Initially, 2-ethylhexyl erucate is first prepared from 2-ethylhexanol (or, under some conditions, 2-ethyl-1-hexene) and erucic acid (not shown). Then, as shown above, the resulting ester is submitted to ozonolysis with reductive quenching using glyoxal, to afford 2-ethylhexyl brassyl aldehyde and nonanal. After removal of the nonanal, the 2-ethylhexyl brassyl aldehyde is oxidized to 2-ethylhexyl brassylate using vanadium pentoxide and air. The resulting ester is hydrolyzed using aqueous sulfuric acid to afford brassylic acid and 2-ethylhexanol, and the latter is recycled to the first step (ester formation).

It is understood that in the recitations of Method 1 and 1.1-1.49, and Method 2 and 2.1-2.53, and Method 3, where the singular terms “fatty acid ester,” “dicarboxylic acid ester,” “erucic acid ester,” “brassylic acid ester,” “oxo-carboxylic acid ester”, and “brassyl aldehyde ester” are used, in some cases, for example where the ester is an isodecyl ester, the ester may be a mixture of structural isomeric esters. Thus, in such a case, the singular case “ester” recited in the above methods should be interpreted as referring to a mixture of isomeric esters.

As used herein, the term “dicarboxylic acid” refers to a linear alkyl dicarboxylic acid having two carboxylic acid groups at opposite ends of the linear alkane chain. Thus, it refers to a dicarboxylic acid of the formula HOOC—(CH₂)ₙCOOH. As used herein, the term “oxo-carboxylic acid” refers to a linear alkyl dicarbonyl compound wherein one end of the linear alkane chain ends in a carboxylic acid group and the other end of the linear alkane chain ends in an aldehyde group. Thus, it refers to a compound of the formula HOOC—(CH₂)ₙCHO. It is further understood that in chemical formulas used herein the group “CHO” or “OHC” refers to an aldehyde group, i.e., —C(═O) H.

As used herein, the term “fatty acid” refers to a saturated or unsaturated, monocarboxylic acid, generally having from 4 to 28 carbon atoms. However, it is also understood that, unless indicated otherwise, the term “fatty acid” refers herein to a monounsaturated or polyunsaturated fatty acid having 7 to 28 carbon atoms.

The term “high melting point dicarboxylic acid” or “high melting dicarboxylic acid” refers herein to a dicarboxylic acid, as defined herein, having a melting point of at least 60° C. The term “high melting point oxo-carboxylic acid” or “high melting oxo-carboxylic acid” refers herein to an oxo-carboxylic acid, as defined herein, having a melting point of at least 50° C.

In a second aspect, the present disclosure provides for use of a C₃₋₁₂ fatty acid ester (e.g., C₃₋₁₂ erucic acid ester) in a process for making a dicarboxylic acid (e.g., brassylic acid), for example, a method according to Method 1 or any of 1.1-1.49, or Method 3, or in a process for making an oxo-carboxylic acid (e.g., brassyl aldehyde), for example, a method according to Method 2 or any of 2.1-2.53, optionally with isolation of the aldehyde by-product (e.g., nonanal).

In a third aspect, the present disclosure provides a dicarboxylic acid (e.g., brassylic acid) made according to Method 1 or any of 1.1-1.49, or Method 3, or an oxo-carboxylic acid (e.g., brassyl aldehyde) made according to Method 2 or any of 2.1-2.53, or an aldehyde (e.g., nonanal) made as a by-product according to Method 2 or any of 2.1-2.53 or Method 3.

In a fourth aspect, the present disclosure provides a product or composition comprising a dicarboxylic acid (e.g., brassylic acid) made according to Method 1 or any of 1.1-1.49, or Method 3, or comprising an oxo-carboxylic acid (e.g., brassyl aldehyde) made according to Method 2 or any of 2.1-2.53. Such products or compositions include, but are not limited to, fragrance, perfume, soap, insect repellent, insecticide, detergent, household cleaning agent, air freshener, room spray, pomander, candle, cosmetic, toilet water, pre- and/or after-shave lotion, talcum powder, haircare product, body deodorant, anti-perspirant, shampoo, pet litter, topical skin care, paint or coating, lubricant, plastic, defoamer, hydraulic fluid, antimicrobial, crop care, and enhanced oil recovery compositions or products.

The term “isodecyl” as used herein refers to any 10-carbon saturated alkyl chain that is not linear (i.e., not n-decyl). Examples of isodecyl groups include, but are not limited to, 2,4-dimethyloctan-2-yl, 2,6-dimethyl-octan-1-yl, 2,6-dimethyloctan-2-yl, 3,7-dimethyloctan-1-yl, and 3,7-dimethyloctan-3-yl.

It is understood that ozonolysis is a two-step process consisting of a first step in which an alkene is reacted with ozone to form a reactive ozonide intermediate (primary and secondary ozonides) and a second step which either oxidizes or reduces the ozonide mixture to provide carbonyl products. The terms “oxidative work-up” and “reductive work-up” refer to the conditions of this second step. It is understood that “oxidative work-up” exposes the reactive ozonides to an oxidizing reagent resulting in carboxylic acid products, while “reductive work-up” exposes the reactive ozonides to a reducing agent resulting in aldehyde products. It is further understood that either or both steps of the ozonolysis, unless provided otherwise herein, may be performed in a batch reactor or in flow reactor.

In some embodiments, traditional reducing agents are used for the “reductive work-up” of the ozonide intermediate mixture. Such agents include, but are not limited to sodium hydroxymethanesulfinate, hydroxymethanesulfinic acid, sulfite salts (e.g., sodium sulfite, sodium bisulfite, sodium metabisulfite, potassium sulfite, potassium bisulfite, potassium metabisulfite), sulfur dioxide, sodium dithionite, dimethyl sulfide, zinc, triphenylphosphine, thiourea, and formic acid, and hydrogenation methods over transition metal catalysts, such as palladium, platinum, and rhodium. However, in some embodiments, the novel ozonide reducing agent glyoxal is employed, optionally as an aqueous solution (e.g., 40% aqueous glyoxal).

### EXAMPLES

### Example 1—Isodecyl Erucate

2,6-dimethyloct-2-ene (14 g, 0.1 mol) is combined with 16.7 g of erucic acid (0.05 mol) in a large glass vial, and the vial is then charged with 1.0 g of Amberlyst H+ catalyst. The reaction is then heated to 40° C. with stirring, melting the erucic acid and forming a single liquid phase. The reaction mixture is stirred for 3 days, until 1H NMR and TLC show the desired product to have formed. The reaction mixture is then diluted with hexane, filtered through a pad of celite and silica to remove all catalyst, and it is then concentrated to remove hexane and residual 2,6-dimethyloct-2-ene. The resulting product, a mixture of 2,6-dimethyloctan-1-yl erucate and 2,6-dimethyl-octan-2-yl erucate, may be used crude in subsequent chemistry or further purified, e.g., via extraction, distillation, and/or chromatography.

### Example 2—2-Ethylhexyl Erucate

Erucic acid (338 g, 1 mol) is combined with 2-ethylhexanol (1.5 mol, 195 g). The mixture is then charged with 9.8 g of concentrated sulfuric acid (0.1 mol) and stirred at 60° C. under vacuum to remove water. After 6 hours of stirring, the reaction is removed from vacuum and heat, and neutralized with 0.1 mol of NaOH (6 M aqueous). The organic mixture is then washed with water and phase separated. The organic phase can then be taken on ‘as-is’, or can be further purified to remove any moisture and excess 2-ethylhexanol under vacuum distillation.

### Example 3—Brassylic Acid

2-Ethylhexyl erucate (450 g, 1 mol) is contacted with 5% ozone in air in a temperature-controlled reactor at 35° C. until ozone consumption ceased and the reaction exotherm abated. 2M aqueous sodium hydroxide (1.5 equiv.) is then added to the reaction mixture slowly with stirring while maintaining the reaction temperature at 60° C. or below. Once the exotherm ceases, the reaction is stirred for an additional 2 hours at 60° C. to ensure completion of the quenching. The reaction is checked by DSC and iodometric titration to confirm quenching. The reaction is then neutralized with 2M aqueous sulfuric acid (1.5 equiv.). The phases are then separated and the organic phase is washed with water, then passed through a wiped film evaporator at reduced pressure to remove trace volatiles and to fraction off the nonanoic acid.

The heavy organic fraction containing the 2-ethylhexyl brassylate intermediate (and 2-ethylhexyl brassyl aldehyde) is then charged with 0.5 g vanadium pentoxide and the reaction is sparged with air at 50° C. for six hours. The mixture is then passed through a Celite filter to remove catalyst.

The filtrate is combined in a 1:1 v/v ratio with water, and then 5% by weight of concentrated sulfuric acid is added. The mixture is stirred vigorously with steam stripping to remove the 2-ethylhexanol as it forms. When the reaction is complete, it is cooled to room temperature and the aqueous phase is filtered to recover brassylic acid crystals.

### Example 5—2-Ethylhexyl Brassyl Aldehyde

2-Ethylhexyl erucate (450 g, 1 mol) is contacted with 5% ozone in air in a temperature-controlled reactor at 35° C. until ozone consumption ceased and the reaction exotherm abated. An aqueous solution of sodium sulfite (1.5 mol equivalents) is then added to the reaction mixture slowly with stirring while maintaining the reaction temperature at 60° C. or below. Once the exotherm ceases, the reaction is stirred for an additional 2 hours at 60° C. to ensure completion of the reductive quenching. The reaction is checked by DSC and iodometric titration to confirm quenching. The phases are then separated and the organic phase is washed with water, then passed through a wiped film evaporator at reduced pressure to remove trace volatiles and to fraction off the nonanal.

### Example 6—2-Ethylhexyl Brassyl Aldehyde via Glyoxal Reduction

2-Ethylhexyl erucate (450 g, 1 mol) is contacted with 5% ozone in air in a temperature-controlled reactor at 35° C. until ozone consumption ceased and the reaction exotherm abated. A 40% aqueous glyoxal solution (1.5 mol glyoxal) is then added to the reaction mixture slowly with stirring while maintaining the reaction temperature at 60° C. or below. Once the exotherm ceases, the reaction is stirred for an additional 2 hours at 60° C. to ensure completion of the reductive quenching. The reaction is checked by DSC and iodometric titration to confirm quenching. The phases are then separated and the organic phase is washed with water, then passed through a wiped film evaporator at reduced pressure to remove trace volatiles and to fraction off the nonanal, thus providing 2-ethylhexyl brassyl aldehyde in good yield and purity.

If desired, standard methods known to the artisan may be employed to convert the 2-ethylhexyl brassyl aldehyde to brassyl aldehyde. For example, a protection-hydrolysis-deprotection route, such as, forming an acetal, followed by base-catalyzed hydrolysis and deprotection of the acetal back to the aldehyde.

The Examples provided herein are exemplary only and are not intended to be limiting in any way to the various aspects and embodiments of the invention described herein.

1. A method for preparing a high melting point dicarboxylic acid from a monounsaturated or polyunsaturated fatty acid, comprising the steps of:
(a) converting the fatty acid to a C₃₋₁₂ fatty acid ester;
(b) performing ozonolysis with oxidative work-up on the C₃₋₁₂ fatty acid ester to form the dicarboxylic acid ester and carboxylic acid by-product; and
(c) converting the dicarboxylic acid ester to the dicarboxylic acid product.

2. The method according to claim 1, wherein the fatty acid has a melting point above 15° C., e.g., above 20° C., or above 25° C., or above 30° C., e.g., up to 60° C.

3. The method according to claim 1, wherein the dicarboxylic acid product has a melting point above 60° C.

4. The method according to claim 1, wherein the fatty acid is selected from erucic acid, palmitoleic acid, oleic acid, elaidic acid, vaccenic acid, gondonic acid, paullinic acid, nervonic acid, stearidonic acid, gamma-linolenic acid, eicosapentaenoic acid, arachidonic acid, docosatetraenonic acid, linolenic acid, linoleic acid, and linoleaidic acid; and optionally, wherein the product dicarboxylic acid is selected from brassylic acid, azelaic acid, adipic acid, glutaric acid, pimelic acid, undecanedioic acid, and pentaundecanedioic acid.

5. A method for preparing brassylic acid from erucic acid, comprising the steps of:
(a) converting erucic acid to a C₃₋₁₂ erucic acid ester, 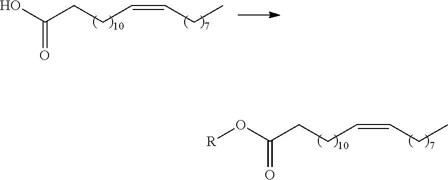
wherein R is a C₃₋₁₂ alkyl;
(b) performing ozonolysis on the C₃₋₁₂ erucic acid ester to form the corresponding brassylic acid ester and nonanoic acid, 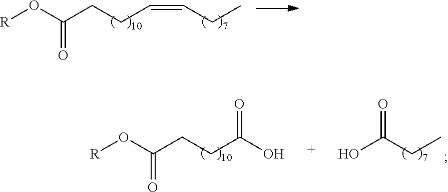
and
(c) converting the brassylic acid ester to brassylic acid (e.g., by aqueous hydrolysis)

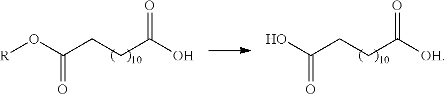

6. The method of claim 5, wherein R is C₃₋₁₀ alkyl.

7. The method of claim 5, wherein R is selected from isopropyl, n-butyl, sec-butyl, isobutyl, tert-butyl, n-pentyl, isopentyl, tert-amyl, n-hexyl, n-heptyl, 2-ethylheptyl, n-octyl, 2-ethylhexyl, n-nonyl, n-decyl, isodecyl.

8. The method of claim 1, wherein the step (b) ozonolysis comprises (1) treating the fatty acid ester with gaseous ozone in a carrier gas in a flow reactor, followed by (2) continuous quenching of the flow reactor product stream under oxidative conditions.

9. The method of claim 8, wherein the step (b)(1) is carried out in a co-current flow reactor.

10. The method of claim 8, wherein the fatty acid ester of step (a) is introduced into the reactor of step (b)(1) as an aqueous emulsion consisting of the neat erucic acid ester and aqueous solution.

11. The method of claim 8, wherein the fatty acid ester of step (a) is introduced into the reactor of step (b)(1) as the neat fatty acid ester.

12. The method of claim 8, wherein step (b)(2) comprising continuously mixing the output stream from the reactor of step (b)(1) with a quenching solution comprising an oxidizing agent.

13. The method of claim 1, wherein step (a) is carried out by reacting the fatty acid with the corresponding C₃₋₁₂ alcohol (R—OH) or C₃₋₁₂ alkene in the presence of an acidic catalyst.

14. The method of claim 1, wherein the product stream from step (b) consists essentially of the dicarboxylic acid ester, the carboxylic acid by-product, and any unreacted fatty acid ester, or carried over fatty acid.

15. The method of claim 1, wherein the product stream from step (b) and/or step (c) does not contain any reduction products.

16. A method for preparing a high melting point oxo-carboxylic acid from a monounsaturated or polyunsaturated fatty acid, comprising the steps of:
(a) converting the fatty acid to a C₃₋₁₂ fatty acid ester;
(b) performing ozonolysis with reductive work-up on the C₃₋₁₂ fatty acid ester to form the oxo-carboxylic acid ester and aldehyde by-product;
and
(c) converting the oxo-carboxylic acid ester to the oxo-carboxylic acid product;
wherein the formation of ozonide intermediate of the ozonolysis step (b) is conducted in a flow reactor.

17. The method according to claim 16, wherein the method further comprises the step of isolating the aldehyde by-product from the crude product, or partially purified product, of step (b) or of step (c).

18. A method for preparing a high melting point dicarboxylic acid from a monounsaturated or polyunsaturated fatty acid, comprising the steps of:
(a) converting the fatty acid to a C₃₋₁₂ fatty acid ester;
(b) performing ozonolysis with reductive work-up on the C₃₋₁₂ fatty acid ester to form the oxo-carboxylic acid ester and aldehyde by-product;
(c) removing the aldehyde by-product;
(d) oxidizing the oxo-carboxylic acid ester to form the dicarboxylic acid ester; and
(e) converting the dicarboxylic acid ester to the dicarboxylic acid-product;
optionally, wherein the formation of ozonide intermediate of the ozonolysis step (b) is conducted in a flow reactor.

19. (canceled)

20. (canceled)

21. (canceled)

22. The method of claim 1, wherein the fatty acid has a melting point of up to 60° C.

23. The method of claim 1, wherein the product stream from step (b) and/or step (c) does not contain any nonanal, nonanol, 13-oxotridecanoic acid, 13-oxotridecanoate esters, 13-hydroxytridecanoic acid, or 13-hydroxytridecanoate esters.
